(19) United States
(12) Patent Application Publication
(10) Pub. No.: US 2017/0080225 A1
(43) Pub. Date: Mar. 23, 2017

(54) SYSTEM FOR ADMINISTERING THERAPEUTIC ELECTRICAL STIMULATION

(71) Applicant: Alton B. Hamm, North Richland Hills, TX (US)

(72) Inventor: Alton B. Hamm, North Richland Hills, TX (US)

(21) Appl. No.: 15/360,796

(22) Filed: Nov. 23, 2016

Related U.S. Application Data
(63) Continuation-in-part of application No. 14/824,849, filed on Aug. 12, 2015.
(60) Provisional application No. 62/185,856, filed on Jun. 29, 2015.

(51) Int. Cl. A61N 1/36; A61N 1/04

(52) U.S. Cl. CPC A61N 1/36014; A61N 1/36003; A61N 1/0452; A61N 1/36021; A61N 1/36007

(57) ABSTRACT

A system and method for treatment using electrical stimulation is described. The electrical stimulation system includes a platform supporting a plurality of electrical conductor handles and a control module. The handles are rigidly coupled to the platform and extend away from a surface. The control module provides a first method of regulating electrical power between the handles. Adjustment of pressure and surface area contact with a conductive matter provides a secondary method and instantaneous electrical power adjustment. Current is passed through conductive matter between the handles. An optional porous compressible member is used to aid in providing satisfactory contact with the conductive matter.

### CLAIM OF PRIORITY

This application claims the benefit of U.S. application Ser. No. 14/824,849, filed 12 Aug. 2015 which claims the benefit of U.S. Provisional Application No. 62/185,856 filed 29 Jun. 2015. The information contained therein is hereby incorporated by reference.

### BACKGROUND

1. Field of the Invention

The present application relates generally to electrical stimulation systems, and more specifically, to a hand held user friendly system for administering therapeutic electrical stimulation to the tissue of the human body.

2. Description of Related Art

Electrical stimulation systems are well known in the art and are effective means to relieve pain by modulating or suppressing pain signals in the brain. For example, FIG. 1 depicts an oblique view of a conventional electrical stimulation system 101 having electrodes 105 and 107 controlled by device 103. During use, and as shown in FIG. 2, electrodes 105 and 107 are adhesively secured on or around an area of pain, such as user's lower back 109, creating a circuit of electrical impulses that stimulate the nerves and modulate or suppress pain signals in the brain.

It should be understood that one of the problems commonly associated with system 101 is that the adhesive used to secure electrodes 105 and 107 leave an unsanitary residue on the user's body after use. Further, system 101 is not user friendly and often requires third party assistance for proper use. It should be also be understood that improper use of system 101 can result in harm or discomfort to the user, for example, when improper contact between electrodes 105 or 107 and the user's lower back 109 creates electrical arcing. Furthermore, adjustment of the electrodes decreases the adhesion properties, therefore finding the right location on the body to apply the electrodes is imperative. Reapplication of electrodes is not always optional. Electrodes then become disposable and create additional costs.

Accordingly, although great strides have been made in the area of relieving pain via electrical stimulation systems, many shortcomings remain.

### DESCRIPTION OF THE DRAWINGS

The novel features believed characteristic of the application are set forth in the appended claims. However, the application itself, as well as a preferred mode of use, and further objectives and advantages thereof, will best be understood by reference to the following detailed description when read in conjunction with the accompanying drawings, wherein:

FIG. 1 is a front view of a conventional therapeutic electrical stimulation system.

FIG. 2 is a front view of the system of FIG. 1 as applied to a user's back.

FIG. 3 is a front view of a user friendly system for therapeutic electrical stimulation in accordance with a preferred embodiment of the present application.

FIG. 4 is a top view of an alternative embodiment of the present application.

FIG. 5 is a control module of the therapeutic electrical stimulation system of FIGS. 3 and 4.

FIG. 6 is a perspective view of another embodiment of the therapeutic electrical stimulation system of FIG. 3.

FIG. 7 is a bottom view of the therapeutic electrical stimulation system of FIG. 6.

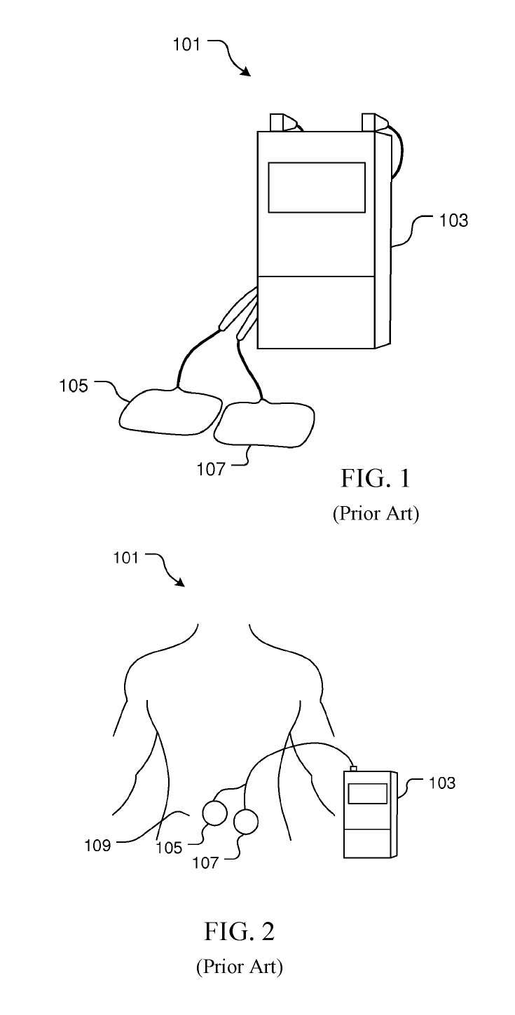

While the system and method of the present application is susceptible to various modifications and alternative forms, specific embodiments thereof have been shown by way of example in the drawings and are herein described in detail. It should be understood, however, that the description herein of specific embodiments is not intended to limit the application to the particular embodiment disclosed, but on the contrary, the intention is to cover all modifications, equivalents, and alternatives falling within the spirit and scope of the process of the present application as defined by the appended claims.

### DETAILED DESCRIPTION OF THE PREFERRED EMBODIMENT

Illustrative embodiments of the preferred embodiment are described below. In the interest of clarity, not all features of an actual implementation are described in this specification. It will of course be appreciated that in the development of any such actual embodiment, numerous implementation-specific decisions must be made to achieve the developer's specific goals, such as compliance with system-related and business-related constraints, which will vary from one implementation to another. Moreover, it will be appreciated that such a development effort might be complex and time-consuming but would nevertheless be a routine undertaking for those of ordinary skill in the art having the benefit of this disclosure.

In the specification, reference may be made to the spatial relationships between various components and to the spatial orientation of various aspects of components as the devices are depicted in the attached drawings. However, as will be recognized by those skilled in the art after a complete reading of the present application, the devices, members, apparatuses, etc. described herein may be positioned in any desired orientation. Thus, the use of terms to describe a spatial relationship between various components or to describe the spatial orientation of aspects of such components should be understood to describe a relative relationship between the components or a spatial orientation of aspects of such components, respectively, as the device described herein may be oriented in any desired direction.

The system and method in accordance with the present application overcomes one or more of the above-discussed problems commonly associated with conventional electrical stimulation systems. In particular, the system is hand held, user-friendly, easy to use, and minimizes the risk of injury, e.g., arcing during use. The system is configured to permit a user the ability to provide varying degrees of electrical current therapy to corresponding portions of the tissue of a human body. The level of current experienced is regulated by the user through a control panel and via the amount of contact (surface area) and amount of pressure instigated by the user. The user operates the system with a single hand and locates a pair of electrodes across different parts of the body as desired. No glues or adhesives are needed to secure stimulating patches. These and other unique features of the device are discussed below and illustrated in the accompanying drawings.

The system and method will be understood, both as to its structure and operation, from the accompanying drawings, taken in conjunction with the accompanying description. Several embodiments of the device may be presented herein. It should be understood that various components, parts, and features of the different embodiments may be combined together and/or interchanged with one another, all of which are within the scope of the present application, even though not all variations and particular embodiments are shown in the drawings. It should also be understood that the mixing and matching of features, elements, and/or functions between various embodiments is expressly contemplated herein so that one of ordinary skill in the art would appreciate from this disclosure that the features, elements, and/or functions of one embodiment may be incorporated into another embodiment as appropriate, unless otherwise described.

The device and method of the present application is illustrated in the associated drawings. The device includes a platform having a plurality of surfaces for the attachment of a control module and a pair of electrical conductors for the generating and dispersing of a current. The electrodes are rigidly affixed to the platform at a particular angle to allow for the ease of spanning a defined area along the conductive matter (i.e. the human body). The electrical conductors together form a positive and negative terminal for the passage of current when both are bridged by a conductive matter. Conductive matter relates to any known matter suited for conducting electrical current, in particular for purposes herein, conductive matter may relate to areas of the human body (i.e. appendages, hands, head . . . ). Other types of matter may be fabrics, meshes, composite materials, and so forth where regulating current may be ideal. The control module permits a user operative control to regulate the dispersion of electrical current through the electrical conductors. Additional features and functions of the device are illustrated and discussed below.

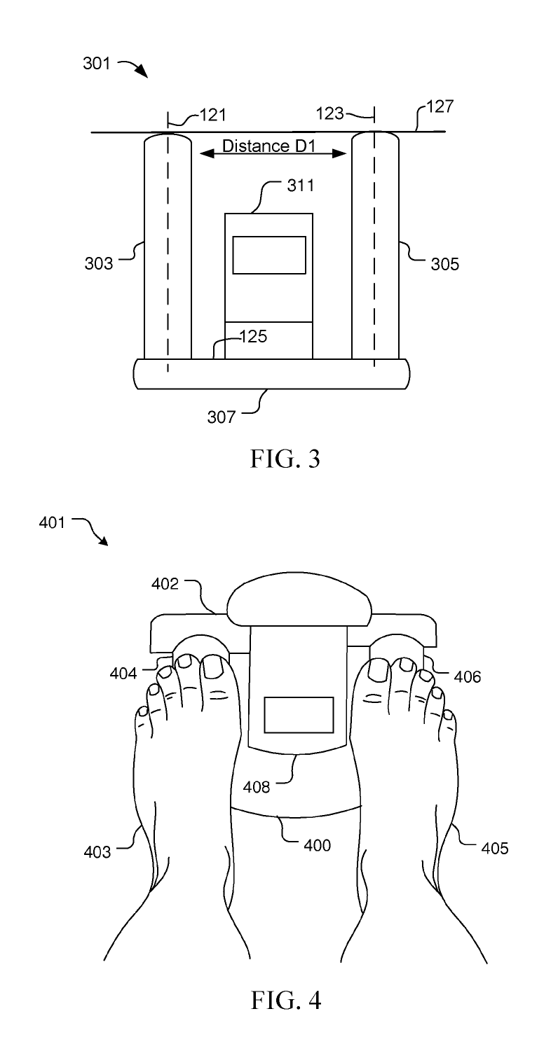

Referring now to the drawings wherein like reference characters identify corresponding or similar elements throughout the several views, FIG. 3 depicts a front view of a user friendly, therapeutic electrical stimulation system 301 in accordance with a preferred embodiment of the present application. It will be appreciated that system 301 overcomes one of more of the above-listed problems commonly associated with conventional electrical stimulation systems.

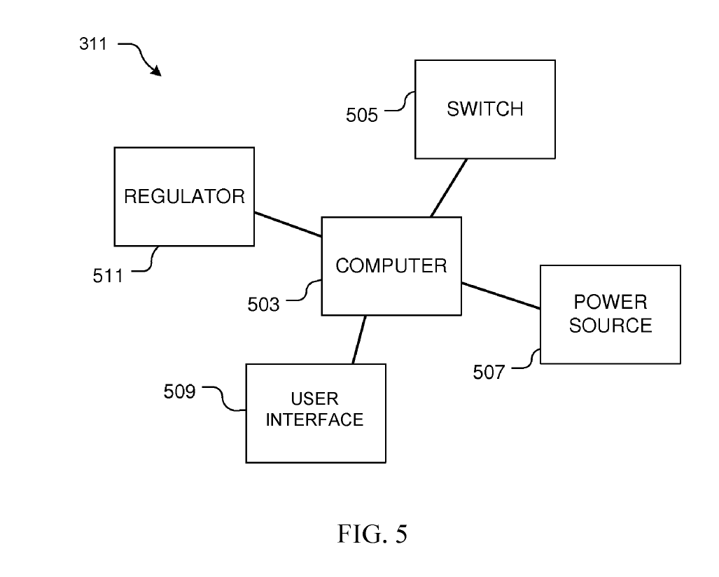

Therapeutic electrical stimulation system of the present application includes a plurality of conductor handles 303, 305 that are rigidly attached to one or more platforms 307. A control module is also included to permit the user to regulate the dispersion of current through the plurality of conductor handles 303, 305. Although not shown, a plurality of conductors and a power source are in electrical communication with handles 303, 305 and a control module 311. Module 311 includes a control system 501 (see FIG. 5) configured to regulate dispersed electrical current from the power source.

Handles 303, 305 may come in many shapes. As illustrated in the drawings, such shape may be that of an elongated cylinder. Each handle 303, 305 is coupled to platform 307 in a rigid manner to prevent undesired flexure and pivoting. As seen in FIG. 3, each handle 303, 305 has an axis 121, 123 respectively. Axes 121 and 123 are configured to be parallel to one another. Additionally, each is perpendicular to platform 307. The two handles 303, 305 are spaced apart from one another a distance D1 and are configured to extend out and away from upper surface 125 of platform 307.

During use, electrical power is passed from power source 507 (see FIG. 5) to handle 303. In this moment, handle 303 is charged. No current flows through handle 303 until distance D1 is spanned by a conductive matter 127. Conductive matter 127 may be any type of material that conducts electricity. Typically this will be considered to be a solid for purposes herein but is understood to also include liquids and combinations of both. Once bridged across distance D1, electrical power is passed through handles 303, 305 and conductive matter 127 to complete the circuit within the system.

Referring now also to FIG. 5 in the drawings, a schematic of control module 311 is illustrated. Control module 311 is configured to regulate the dispersion of electrical current from the power source into handle 303. Module 311 includes a regulator 511, a switch 505, power source 507 and a user interface 509. Control module 311 is configured to regulate an amount and nature of electrical energy to the handle 303. This is performed by selectively permitting and adjusting the amount of energy passing from power source 507. Regulator 511 acts as a gate to allow a user the ability to adjust any of the current level, frequency, and voltage of the electrical power. In particular, this is done via user interface 509. Interface 509 includes one or more buttons, switches, dials, knobs, and so forth to allow the user incremental control of the electrical power. Additionally, interface 509 may allow the user to visually inspect the amount of electrical energy transferred to the handles 303, 305 (i.e. a display). Furthermore, module 311 is configure to indicate to the user either or both expected output level and current real-time output levels.

Module 311 includes a computer 503 having the necessary hardware and software to control the electrical energy and operably associated with a switch 505, power source 507, display 509, and regulator 511. Switch 505 is configured to cut off the flow of power to handle 303 as desired. Computer 503 is in communication with each of the other components within module 311.

Apart from computer 503 and module 311, the level of electrical power passing through handles 303, 305 may alternatively be regulated by the user through the interaction of handles 303, 305 and conductive matter 127. The levels of electrical power may be increased and decreased through changing the amount of surface area contact between handles 303, 305 and conductive matter 127. Additionally, the level of electrical power may be increased and decreased through changing the pressure applied to the conductive matter 127 from either of handles 303, 305. In this way, a user is able to instantaneously regulate electrical power levels without the use of control module 311. Slight movements of the user provide this secondary control method for regulating electrical power. This is especially useful where this handheld portable system is oriented such that the user is not able to access or see user interface 509. Additionally, this secondary control method allows for instantaneous and infinite control as opposed to user interface 509.

It should be appreciated that one of the unique features believed characteristic of the present application is the ease of use. For example, system 301 eliminates any need for wires to be applied to areas on the body that are difficult to reach. Further, there is no need for adhesive materials to be applied to the body, which in turn leave a residue. In the contemplated embodiment, the user merely manipulates the voltage output via control module 311 or through surface area and pressure adjustments to complete the electrical circuit.

The application of electrical stimulation can serve many benefits and accomplish many different tasks. One particular application is in the treatment of the human body. The human body can be considered a conductive matter, such that contacting the body to handles 303 and 305 so as to bridge distance D1 provides a stimulating current of electrical power through are selected portion of the body. The current is isolated to portions of the conductive matter spanning between handles 303, 305. For example, if handles 303, 305 were both pressed to the same thigh, the electrical stimulation would remain generally within the thigh. Conversely, if handles 303, 305 were spanning different appendages (i.e. arm and leg) then the electrical stimulation would route between those appendages over a larger area.

It will be appreciated that handles 303, 305 can be applied to any 2 appendages of the body to create a current. Unlike a conventional therapeutic electrical system, system 301 contemplates that the intensity of the current can be controlled in part through the degree of contact with the conductive matter. For example, the user can firmly grip the handles to increase the electrical contact (increase pressure). Also by adjusting the grip, the surface area contact with handles 303, 305 changes resulting in modified electrical power. Similarly, it is also contemplated and will be appreciated that the electrical resistance of an appendage in system 301 can be decreased to increase the intensity of the electrical contact. For example, a user can wear wet gloves or socks to both increase and more efficiently feel the effects of an electrical contact with system 301 along the length of the appendage.

Electrical stimulation has been found to have medical benefits. It is useful in treating many types of ailments, namely muscular ailments. Ailments of the human body, treatable through system 301, are at least the following vertigo, incontinence, ringing in the ears, migraines, swallowing, and bowel control. Broadly speaking, any portion of a body associated with the nervous system is treatable through system 301. System 301 is configured to be used at any two locations on a human body. Exemplary locations may include, feet, head, legs, ears, and so forth. System 301 allows for the immediate adjustment in location on conductive matter 127 too. Whereas electrode patches are temporarily stuck at a single location, handles 303, 305 are instantaneously adjustable to any location at any moment. Electrical current travels from handle 303 to the hands (not shown) and through the user's body to handle 305, which grounds the current. As the current passes through the user's body it stimulates or modulates a variety of therapeutic biological responses, such as suppressing pain signals in the brain, improving hormone production, and stimulating blood flow.

Referring now also to FIG. 4 in the drawings, a therapeutic electrical system 401 is shown in accordance with an alternative embodiment of the present application. It will be appreciated that system 401 is substantially similar in form and function to system 301 and includes one or more of the features discussed herein.

In this embodiment, system 401 includes one or more of a platform 400 for the user to stand upon and an elongated member 402 rigidly attached to and extending in an upright position relative to the platform 400. One of the unique features of system 401 is the ability to perform the functions of system 301 with the feet 403, 405. To achieve this feature, system 401 includes a first electrical conductor 404 spaced apart from a second conductor 406; both conductors being in electrical communication with device 408.

During use, the user stands on platform 400 and places one or more toes on the electrical conductors. The electrical energy is channeled to the feet via the conductors and an electrical circuit is completed. In the preferred embodiment, the electrical conductors are configured with contouring to match the contouring of the user's toes.

Accordingly, the features of systems 301, 401 allow the user to complete an electrical circuit via the hands or feet, or any other conductive matter, as shown in the figures and discussed above. It is understood that the body is not the only conductive matter usable with systems 301 and 401. They serve as an exemplary manner of use.

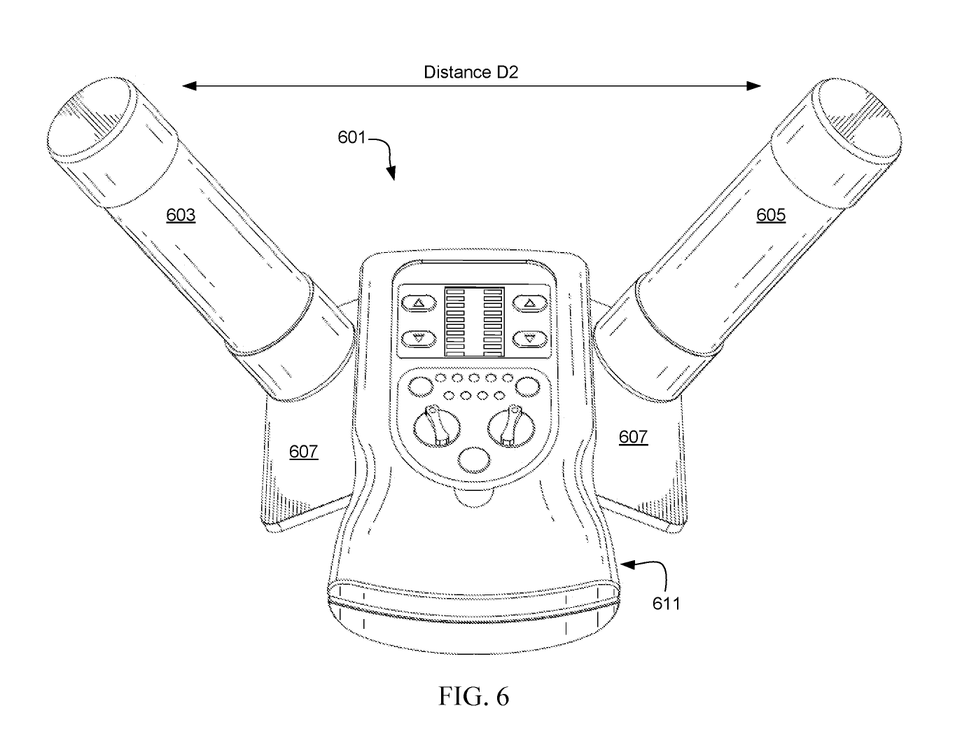

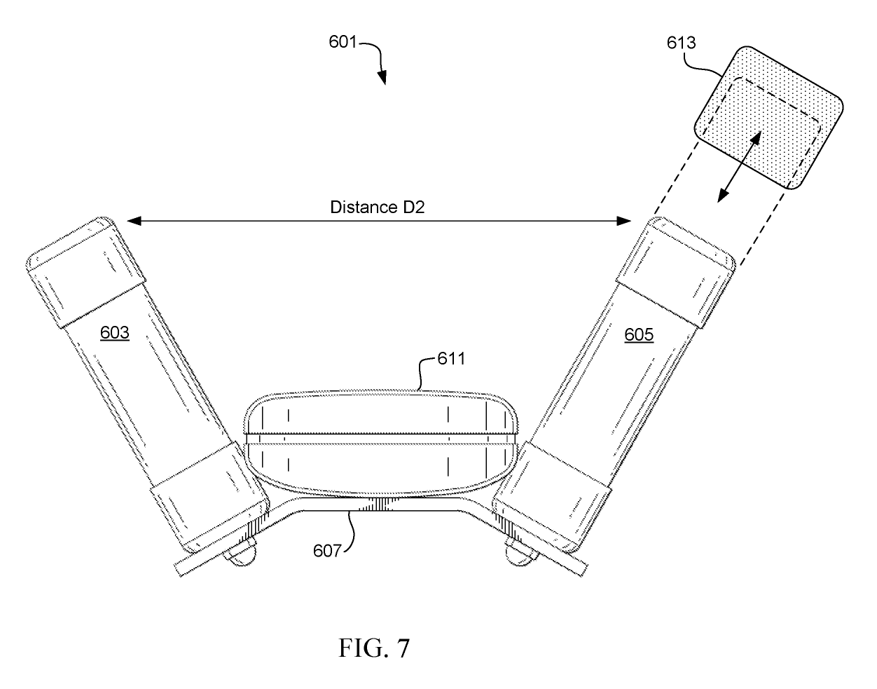

Referring now also to FIGS. 6 and 7 in the drawings, another alternative embodiment of system 301 is illustrated. It will be appreciated that system 601 is substantially similar in form and function to system 301 and includes one or more of the features discussed herein. Control module 611 is similar in form and function to that of module 311.

A predominant difference between system 301 and system 601 is the angle of orientation of the electrical conductor handles. System 601 includes electrical conductor handles 603, 605 similar in form and function to that of handles 303, 305. Platform 607 is configured to angle handles 603, 605 away from one another in a non-parallel orientation. This orientation allows for a greater distance between distal ends of handles 603, 605, such that distance D2 is larger than distance D1. Each is still coupled perpendicularly to platform 607.

FIG. 6 shows a bottom view of system 601. Additionally, FIG. 7 introduces a new optional member of system 501. This is also equally available for use with system 301. A porous compressible member 613 is optionally included such that it is configured to surround a portion of either handle 603 or 605. At times, excess material debris can prevent good contact between handles 603, 605 and conductive matter 127. Member 613 is shown as a hollowed member configured to slide over the distal end of handle 605. As a porous compressible member, member 613 is configured to absorb a liquid and become saturated. A user may elect to saturate member 613 and selectively release the liquid over the conductive matter 127 by compressing member 613. The liquid helps to improve conductive contact with matter 127. Examples of use may include an instance where handle 605 is contacting the scalp and the user's hair prevents good contact. Saturating the scalp using member 613 allows the liquid to carry the electrical power through the hair and to the scalp. This same principle works with clothing. An example of member 613 can be a sponge. Other materials are also contemplated. Member 613 may be applied or removed as desired.

The particular embodiments disclosed above are illustrative only and are not intended to be exhaustive or to limit the invention to the precise form disclosed, as the embodiments may be modified and practiced in different but equivalent manners apparent to those skilled in the art having the benefit of the teachings herein. It is therefore evident that the particular embodiments disclosed above may be altered or modified, and all such variations are considered within the scope and spirit of the application. Accordingly, the protection sought herein is as set forth in the description. It is apparent that an application with significant advantages has been described and illustrated. Although the present application is shown in a limited number of forms, it is not limited to just these forms, but is amenable to various changes and modifications without departing from the spirit thereof.

What is claimed is:

1. An electrical stimulation system, comprising:
a platform having a plurality of surfaces;
a first electrical conductor handle coupled to the platform;
a second electrical conductor handle coupled to the platform, the two electrical conductor handles being spaced apart from one another, both extending out and away from an upper surface of the platform;
an electrical power source; and
a control module configured to regulate the dispersion of electrical current from the power source into the first electrical conductor handle;
wherein electrical power passes from the first electrical conductor handle to the second electrical conductor handle when bridged by a conductive matter.

2. The system of claim 1, wherein the control module is configured to regulate an amount and nature of electrical energy to the first electrical conductor.

3. The system of claim 1, the control module includes a user interface configured to communicate the amount and nature of electrical energy provided to the first electrical conductor.

4. The system of claim 1, the control system, having:
a regulator configured to regulate an amount and nature of electrical energy to the first electrical conductor.

5. The system of claim 1, wherein the first and second electrical conductor handles are parallel and both perpendicular to the upper surface.

6. The system of claim 1, wherein the first and second electrical conductor handles are angled relative to one another so as to be non-parallel.

7. The system of claim 1, further comprising:
a porous compressible material in communication with a portion of the first electrical handle.

8. The system of claim 7, wherein the porous compressible material is configured to selectively absorb and release a liquid.

9. A method of treatment through stimulation of muscular tissue, comprising:
providing the system of claim 1;
regulating an amount and nature of electrical energy passing to the first electrical conductor handle;
contacting the first electrical conductor handle to a first location on a conductive matter; and
contacting the second electrical conductor to a second location on the conductive matter;
wherein electrical power is passed through the conductive matter between the first and second electrical conductor handles such that the muscular tissue is stimulated.

10. The method of claim 9, further comprising:
displaying the amount and nature of electrical energy passing through the first electrical conductor and the second electrical conductor via a user interface.

11. The method of claim 9, wherein the stimulated muscular tissue is configured to alleviate and treat human based muscular ailments.

12. The method of claim 9, wherein the muscular ailments can be at least any of vertigo, incontinence, ringing in the ears, migraines, swallowing, and bowel control.

13. The method of claim 9, further comprising:
adjusting the electrical power to the first electrical conductor handle by changing the contact surface area of either of the first and second electrical conductor handles with the conductive matter.

14. The method of claim 9, further comprising:
adjusting the electrical power to the first electrical conductor handle by changing the pressure applied to the conductive matter by at least one of the first and the second electrical conductor handles.

15. The method of claim 9, wherein adjusting the level of electrical power is performed without use of the control module.

16. The method of claim 9, wherein the conductive matter is the human body.

17. The method of claim 9, further comprising:
applying a porous compressible member to an end of the first electrical conductor handle.

18. The method of claim 17, wherein the porous compressible member is a sponge.

19. The method of claim 17, further comprising:
saturating the porous compressible member.

20. The method of claim 19, further comprising:
compressing the porous compressible member so as to release the liquid, the liquid configured to conduct electrical power.
